(19) United States
(12) Patent Application Publication
(10) Pub. No.: US 2018/0049694 A1
(43) Pub. Date: Feb. 22, 2018

(54) SYSTEMS AND METHODS FOR DETERMINING INDIVIDUALIZED ENERGY EXPENDITURE

(71) Applicant: Apple Inc., Cupertino, CA (US)

(72) Inventors: Alexander Singh Alvarado, Mountain View, CA (US); Craig H. Mermel, San Jose, CA (US); Karthik Jayaraman Raghuram, Santa Clara, CA (US); Mrinal Agarwal, Cupertino, CA (US)

(73) Assignee: Apple Inc., Cupertino, CA (US)

(21) Appl. No.: 15/678,645

(22) Filed: Aug. 16, 2017

Related U.S. Application Data
(60) Provisional application No. 62/375,485, filed on Aug. 16, 2016.

(51) Int. Cl. A61B 5/00; A61B 5/024; A61B 5/11

(52) U.S. Cl. CPC A61B 5/4866; A61B 5/02438; A61B 5/02416; A61B 5/1112; A61B 2503/10; A61B 5/681; A61B 5/0022; A61B 2560/0242; A61B 5/1116

(57) ABSTRACT

A method and a system for determining an individual energy expenditure are described. In some embodiments, an energy expenditure can be calculated based on a combination of biometrics, heart rate and work rate. In some embodiments, a relative drag associated with the user can be calculated based on a group formation size, a group formation shape, participant velocities, weather, air density, and participant body surface areas. In some embodiments, a load adjustment factor can be determined based on the relative drag. In some embodiments, an adjusted energy expenditure can be determined based on the load adjustment factor.

### CROSS-REFERENCE TO RELATED APPLICATION

This application claims priority to and the benefit of U.S. Provisional Application No. 62/375,485 filed on Aug. 16, 2016, the disclosure of which is incorporated by reference herein in its entirety.

### FIELD

The present disclosure relates generally to improving calorie expenditure prediction and tracking and, more particularly, to techniques for determining energy expenditure factoring in drag in group sporting activities.

### BACKGROUND

Current calorie prediction devices are not able to take the context of a cyclist into account when computing energy expenditure. For example, cyclists riding in groups often take advantage of formations to optimize their speeds. Each rider in a formation experiences different loads due to drafting, tail winds or head winds which in turn affects the energy expenditure of each participant.

### SUMMARY

The present disclosure relates to a method for improving the accuracy of a wearable device while calculating an individual energy expenditure for a user participating in a group cycling session. In one aspect, the method can include measuring a hear rate of the user with a heart rate sensor, wherein the heart rate sensor comprises a photoplethysmogram (PPG) sensor and the PPG sensor is configured to be worn adjacent to the user's skin; calculating an energy expenditure of the user based on the measured heart rate of the user; determining a group formation size and a group formation shape based on wireless-based proximity; receiving a wind speed and direction from an external source; determining the user's velocity; determining an air density based on an ambient temperature and an atmospheric pressure; determining a relative drag associated with the user based on the group formation size, the group formation shape, the user's velocity, the user's body surface area, the wind speed and direction, and the air density; calculating a load adjustment factor based on the determined relative drag; determining an updated energy expenditure based on the calculated energy expenditure and the load adjustment factor; and outputting the updated energy expenditure.

In some embodiments, the method can also include determining a cross sectional area of the user based on the user's body surface area and the user's posture.

In some embodiments, the method can also include determining a number and a relative position of nearby devices; determining a group formation size based on the number of nearby devices; and determining a group formation shape based on the relative position of nearby devices.

In some embodiments, the method can include determining a number of devices that have successfully connected to the wearable device wirelessly. In some embodiments, the method can include determining a number of devices within a pre-defined distance from the wearable device. In some embodiments, the method can include performing time of flight calculations, measuring wireless signal strength of nearby devices, or using multiple directional antennas.

In some embodiments, the method can also include deriving a maps of devices from the relative position of nearby devices; performing a matching between the derived map of devices and one or more known group formation shapes; and determining a group formation shape based on a result of the matching. In some embodiments, the known group formation shapes can include a straight line, a cluster, echelon, single paceline, double paceline, circular paceline, or V formation.

The present disclosure also relates to a system for improving the accuracy of a wearable device while calculating an individual energy expenditure for a user participating in a group cycling session. In one aspect, the system can include a heart rate sensor configured to measure a heart rate of the user, wherein the heart rate sensor comprises a photoplethysmogram (PPG) sensor and the PPG sensor is configured to be worn adjacent to the user's skin; a GPS module configured to measure the user's location and velocity; a wireless module configured to measure a wireless-based proximity of nearby devices; and a processor circuit coupled to all the modules. In some embodiments, the processor circuit can calculate an energy expenditure based on at least the measured heart rate; determine a group formation size and a group shape based on the measured wireless-based proximity; determine an air density based on an ambient temperature and an atmospheric pressure; receive a wind speed and direction from an external source; determine a relative drag associated with the user based on the group formation size, the group formation shape, the user's velocity, the user's body surface area, the wind speed and direction, and the air density; calculate a load adjustment factor based on the determined relative drag; determine an updated energy expenditure based on the calculated energy expenditure and the load adjustment factor; and output the updated energy expenditure.

In some embodiments, a motion sensing module of the wearable device can detect the user's posture. The processor circuit can determine a cross sectional area of the user based on the user's body surface area and the user's posture.

In some embodiments, the processor circuit can determine a number and a relative position of nearby devices; determine a group formation size based on the number of nearby devices; and determine a group formation shape based on the relative position of nearby devices.

In some embodiments, the processor circuit can determine a number of devices that have successfully connected to the wearable device wirelessly. In some embodiments, the processor circuit can determine a number of devices within a pre-defined distance from the wearable device.

In some embodiments, the processor circuit can derive a map of devices from the relative position of nearby devices; perform a matching between the derived map of devices and one or more known group formation shapes; and determine a group formation shape based on a result of the matching. In some embodiments, the know group formation shapes can include a straight line, a cluster, echelon, single paceline, double paceline, circular paceline, or V formation.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 shows an example of a fitness tracking device (or a “wearable device”) 100, according to some embodiments of the present disclosure.

FIG. 2 depicts a block diagram of example components that may be found within the fitness tracking device 100, according to some embodiments of the present disclosure.

FIG. 3 shows an example of a companion device 300, according to some embodiments of the present disclosure.

FIG. 4 is a diagram showing the calculation of an adjusted energy expenditure, according to some embodiments of the present disclosure.

FIG. 5 is a flowchart showing a process of determining size and shape of a cycling group, according to some embodiments of the present disclosure.

FIG. 6 is a flowchart showing a process for determining energy expenditure, according to some embodiments of the present disclosure.

### DETAILED DESCRIPTION

Systems and methods are disclosed herein to optimize calorie expenditure predictions by taking into account drag, head winds or tail winds, which can be affected by a cycling group's formation size and shape. The systems and methods disclosed herein also include mapping a cycling group's formation size and shape by using a combination of device proximity data, location data, cyclists' biometric data and environmental conditions.

In some embodiments, the systems and methods disclosed herein can also be used to determine the contribution of each cyclist to a cycling group. The determination of contribution can be used to adjust a cyclist's position in the group to maximize a speed of the group (e.g., in peloton cycling) or to normalize users' contributions to the cycling group.

In some embodiments, the systems and methods disclosed herein can also be used with other sports where people move in a group (e.g., a pack of runners or swimmers). Additionally, the systems and methods disclosed herein can also be used with an autonomous vehicle platform to optimize energy efficiency while traveling.

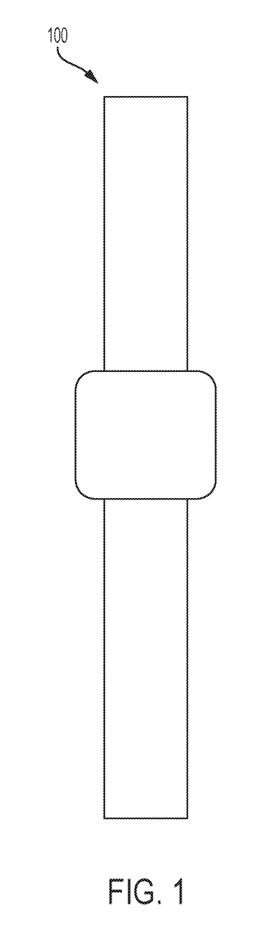

FIG. 1 shows an example of a fitness tracking device 100 (or “a wearable device”), according to some embodiments of the present disclosure. In some embodiments, the fitness tracking device 100 may be a wearable device, such as a watch configured to be worn around an individual's wrist. As described in more detail below, the fitness tracking device 100 may be calibrated according to physical attributes of the individual and physical activity by the individual user who is wearing the fitness tracking device 100, including, for example, heart rate statistics.

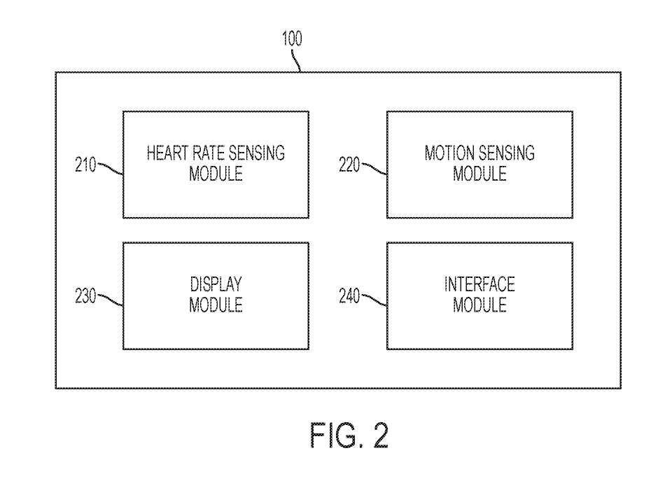

FIG. 2 depicts a block diagram of example components that may be found within the fitness tracking device 100, according to some embodiments of the present disclosure. These components may include a heart rate sensing module 210, a motion sensing module 220, a display module 230, and an interface module 240.

The heart rate sensing module 210 may include or may be in communication with a photoplethysmogram “PPG” sensor as previously described. The fitness tracking device 100 can measure an individual's current heart rate from the PPG. The heart rate sensor may also be configured to determine a confidence level indicating a relative likelihood of an accuracy of a given heart rate measurement. In other embodiments, a traditional heart rate monitor may be used and may communicate with the fitness tracking device 100 through a communication method (e.g., Bluetooth®).

The fitness tracking device 100 may include an LED and a photodiode or the equivalent to obtain a PPG. The fitness tracking device 100 may subsequently determine the user's current heart rate based on the PPG data.

To conserve battery power on the fitness tracking device 100, the LED may be a relatively low-power LED, such as a green LED. In some embodiments, to further conserve power on the fitness tracking device 100, the fitness tracking device 100 may be configured to check heart rate at periodic intervals (e.g., once per minute, or once per three minutes). The period for checking heart rate may change dynamically. For example, if the fitness tracking device 100 automatically detects or receives input from the user that the user is engaged in a certain level, intensity, or type of physical activity (e.g., “in session”), the fitness tracking device may check heart rate more frequently (e.g., once per thirty seconds, once per minute, etc.). The fitness tracking device 100 may use, for example, machine learning techniques, battery power monitoring, or physical activity monitoring to balance the frequency of heart rate samples for accurate calorimetry with power optimization.

In addition to the heart rate sensing module 210, the fitness tracking device 100 may also include the motion sensing module 220. The motion sensing module 220 may include one or more motion sensors, such as an accelerometer or a gyroscope. In some embodiments, the accelerometer may be a three-axis, microelectromechanical system (MEMS) accelerometer, and the gyroscope may be a three-axis MEMS gyroscope. A microprocessor (not shown) or motion coprocessor (not shown) of the fitness tracking device 100 may receive motion information from the motion sensors of the motion sensing module 220 to track acceleration, rotation, position, or orientation information of the fitness tracking device 100 in six degrees of freedom through three-dimensional space.

In some embodiments, the motion sensing module 220 may include other types of sensors in addition to accelerometers and gyroscopes. For example, the motion sensing module 220 may include an altimeter or barometer, or other types of location sensors, such as a GPS sensor or a Bluetooth® sensor. A barometer (also referred to herein as a barometric sensor) can detect pressure changes and correlate the detected pressure changes to an altitude.

In some embodiments, the fitness tracking device 100 may take advantage of the knowledge that the heart rate sensing module 210 and the motion sensing module 220 are approximately co-located in space and time to combine data from each module 210, 220 to improve the accuracy of its calorimetry functionality. Depending on the current activity and a determination of a confidence of current heart rate and motion data, the fitness tracking device 100 may also rely on one of either the heart rate or a motion-derived work rate to estimate energy expenditure more accurately.

The fitness tracking device 100 may also include a display module 230. Display module 230 may be a screen, such as a crystalline (e.g., sapphire) or glass touchscreen, configured to provide output to the user as well as receive input form the user via touch. For example, display 230 may be configured to display a current heart rate or a daily average energy expenditure. Display module 230 may receive input from the user to select, for example, which information should be displayed, or whether the user is beginning a physical activity (e.g., starting a session) or ending a physical activity (e.g., ending a session), such as a running session or a cycling session. In some embodiments, the fitness tracking device 100 may present output to the user in other ways, such as by producing sound with a speaker (not shown), and the fitness tracking device 100 may receive input from the user in other ways, such as by receiving voice commands via a microphone (not shown).

In some embodiments, the fitness tracking device 100 may communicate with external devices via interface module 240, including a configuration to present output to a user or receive input from a user. Interface module 240 may be a wireless interface. The wireless interface may be a standard Bluetooth® (IEEE 802.15) interface, such as Bluetooth® v4.0, also known as “Bluetooth® low energy.” In other embodiments, the interface may operate according to a cellphone network protocol such as LTE or a Wi-Fi (IEEE 802.11) protocol. In other embodiments, interface module 240 may include wired interfaces, such as a headphone jack or bus connector (e.g., Lightning, Thunderbolt, USB, etc.).

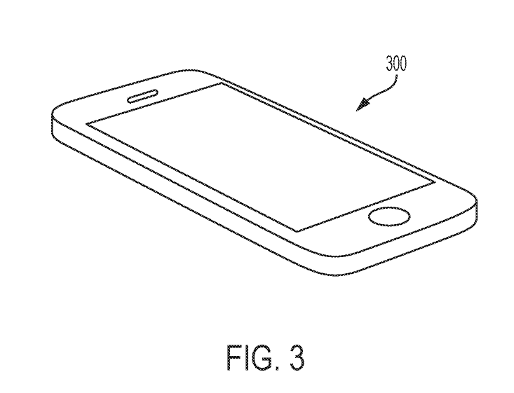

The fitness tracking device 100 may be configured to communicate with a companion device 300 (FIG. 3), such as a smartphone, as described in more detail herein. In some embodiments, the fitness tracking device 100 may be configured to communicate with other external devices, such as a notebook or desktop computer, tablet, headphones, Bluetooth® headset, another fitness tracking device, etc.

In some embodiments, the fitness tracking device 100 can have one or more antennas. In some embodiments, having more than one antenna (e.g., two antennas) can improve both Wi-Fi and Bluetooth®-based directionality sensing. In some embodiments, the antennas can be directional antennas.

The modules described above are examples, and embodiments of the fitness tracking device 100 may include other modules not shown. For example, the fitness tracking device 100 may include one or more microprocessors (not shown) for processing heart rate data, motion data, other information in the fitness tracking device 100, or executing instructions for firmware or apps stored in a non-transitory processor-readable medium such as a memory module (not shown). Additionally, some embodiments of the fitness tracking device 100 may include a rechargeable battery (e.g., a lithium-ion battery), a microphone or a microphone array, one or more cameras, one or more speakers, a watchband, a crystalline (e.g., sapphire) or glass-covered scratch-resistant display, water-resistant casing or coating, etc.

FIG. 3 shows an example of a companion device 300, according to some embodiments of the present disclosure. The fitness tracking device 100 may be configured to communicate with the companion device 300 via a wired or wireless communication channel (e.g., Bluetooth®, Wi-Fi, etc.). In some embodiments, the companion device 300 may be a smartphone, tablet, or similar portable computing device. The companion device 300 may be carried by the user, stored in the user's pocket, strapped to the user's arm with an armband or similar device, placed on a table, or otherwise positioned within communicable range of the fitness tracking device 100.

The companion device 300 may include a variety of sensors, such as location and motion sensors (not shown). When the companion device 300 may be optionally available for communication with the fitness tracking device 100, the fitness tracking device 100 may receive additional data from the companion device 300 to improve or supplement its calibration or calorimetry processes. For example, in some embodiments, the fitness tracking device 100 may not include a GPS sensor as opposed to an alternative embodiment in which the fitness tracking device 100 may include a GPS sensor. In the case where the fitness tracking device 100 may not include a GPS sensor, a GPS sensor of the companion device 300 may collect GPS location information, and the fitness tracking device 100 may receive the GPS location information via interface module 240 (FIG. 2) from the companion device 300.

In another example, the fitness tracking device 100 may not include an altimeter, as opposed to an alternative embodiment in which the fitness tracking device 100 may include an altimeter. In the case where the fitness tracking device 100 may not include an altimeter or barometer, an altimeter or barometer of the companion device 300 may collect altitude or relative altitude information, and the fitness tracking device 100 may receive the altitude or relative altitude information via interface module 240 (FIG. 2) from the companion device 300.

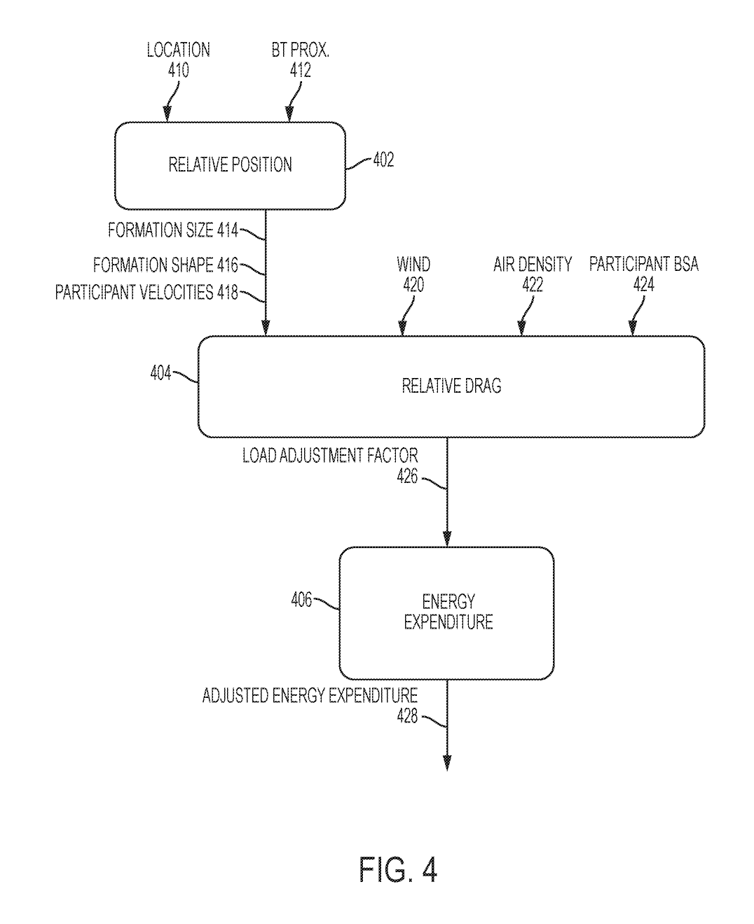

FIG. 4 is a diagram showing the calculation of an adjusted energy expenditure, according to some embodiments of the present disclosure. FIG. 4 shows relative position 402, relative drag 404, energy expenditure 406, location 410, Bluetooth® proximity 412, formation size 414, formation shape 416, participant velocities 418, wind 420, air density 422, participant body surface areas (BSAs) 424, load adjustment factor 426, and adjusted energy expenditure 428.

As shown in FIG. 4, and as described in more detail below, determining an adjusted energy expenditure depends on location 410, Bluetooth® proximity 412, weather 420, air density 422, and participant body surface areas (BSAs) 424. In some embodiments, each of location 410, Bluetooth® proximity 412, weather 420, and air density 422 are measured at time intervals by one or more of the fitness tracking device 100 and the companion device 300. The time interval (e.g., every second, ten times a second) can be a default value set by an administrator. The time interval can also be adjusted by an administrator or adapt to sensed network or environmental conditions. In some embodiments, instead of Bluetooth®, other wireless-based proximity can also be used, such as Wi-Fi.

In some embodiments, the start of a session for determining energy expenditure as described herein can be started ad hoc by a user, started according to a scheduled time, or started based on one or more sensed parameters of the user or his/her environment (e.g., accelerated heart rate, temperature change, change in velocity and proximity to others).

Three of the components used to determine an adjusted energy expenditure include a relative position 402, a relative drag 404, and an energy expenditure adjustment 406. Relative position 402 is a distance between one cyclist and another cyclist and be continuously measured and calculated for an exercise session. In some embodiments, a relative position 402 can be determined using both location services 410 (e.g., GPS, cell towers, Wi-Fi) and Bluetooth®-based proximity 412. Location services 410 can be used to determine a velocity of a user and of other cyclists in a pack also wearing a comparable fitness tracking device 418 and Bluetooth®-based proximity 412 can be used to determine a cycling group formation shape 416 and size 414. In some embodiments, instead of Bluetooth®, other wireless-based proximity can also be used. For example, in some embodiments, a wearable device can communicate with a device associated with another user in the group (e.g., through a third party application such as Strava®) to get the position information of other cyclists. A cycling group formation shape 416 refers to an arrangement of the cyclists in the group. Shapes can include a straight line, a cluster, echelon, single paceline, double paceline, circular paceline, and V formation. A cycling group size 414 refers to a number of cyclists in the group.

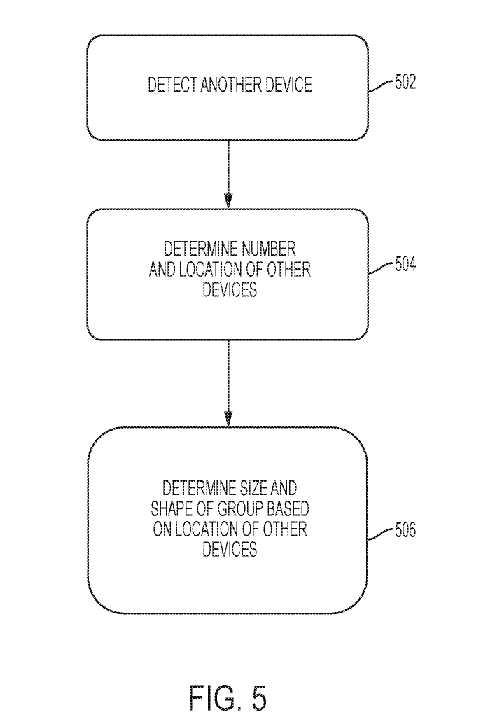

FIG. 5 is a flowchart showing a process of determining size and shape of a cycling group, according to some embodiments of the present disclosure.

Referring to step 502, one or more of a fitness tracking device 100 and companion device 300 detects another device within proximity of one or more of the fitness tracking device 100 and companion device 300. Detection can include using both location services 410 and Bluetooth®-based proximity 412. For example, time of flight calculations between devices and received signal strength indicator (RSSI) levels can be used to determine proximity. In some embodiments, other wireless-based proximity can also be used. Additionally, for higher confidence proximity, a “group” workout mode can be enabled to whitelist devices of interest in the formation to more accurately track the proximity. Antenna specifications can also help with detection (e.g., using directional antennas).

Referring to step 504, based on the proximity information, a number of and location of other devices is determined. Determining a number of devices can include determining a number of devices that have successfully connected via Bluetooth® at least one of the fitness tracking device 100 and companion device 300. In some embodiments, determining a number of devices can include determining a number of devices within a certain distance from at least one of the fitness tracking device 100 and companion device 300. In some embodiments, one or more antennas can be used to detect the relative positions of one or more cyclists in the group.

Referring to step 506, a size and shape of the cycling group are determined based on the location and number of the other devices. Determining formation size and shape can be accomplished using relative location of devices in relation to one another. A single device can use its multiple directional antennas and Bluetooth® signal strength of nearby devices to get the position of other devices around it. In some embodiments, other wireless signal strength can be used, such as Wi-Fi. Polling all the devices part of the formation for the relative locations of nearby devices can form a map of devices.

Once a map is derived of all devices within the formation, a matching can be performed between the detected formation shape and a bank of known formations. In general, detected formation shape can be subject to inaccuracies from sensor variation and environmental attenuation. This makes classifying detected formation against predefined formations useful. It also helps determine what drag coefficients to factor into the analysis.

If a formation shape and size cannot be satisfactorily classified, a relative position of nearby devices at an individual level can always be used to determine drag coefficients.

Referring to FIG. 4, drag refers generally to an amount of resistance. As used herein, relative drag 404 refers to an amount of force acting against the direction of motion of the user. The relative drag 404 can be either positive (e.g., exerting force against the direction of motion and slowing down a speed associated with the direction of motion) or negative (e.g., exerting force in the same direction as the direction of motion and accelerating a speed associated with the direction of motion). Relative drag 404 can be determined based on formation size 414, formation shape 416, participant velocities 418, weather 420, air density 422, and participant BSA 424.

Wind 420 as used herein refers primarily to wind speed and direction. Wind blowing in a direction generally opposite of a direction of travel can cause positive drag, while wind blowing in a direction generally aligned with a direction of travel can cause negative drag. Cross winds can also have similar effects depending on the x-y components of the wind. The wind speed and direction can be received by fitness tracking device 100 and/or companion device 300 from an external source (e.g., Internet, cellular network, etc.)

Air density 422 is directly proportional to drag (e.g., with all other factors being equal, the higher the air density, the higher the drag). Air density 422 depends on temperature, pressure, and humidity. Temperature is inversely proportional to air density 422 (e.g., with all other factors being equal, the higher the temperature, the lower the air density). Pressure is directly proportional to air density and humidity is inversely proportional to air density. For example, air density, and therefore drag, is generally higher at lower temperatures, lower elevations, and at lower levels of humidity.

Participant BSA 424 as used herein refers to the body surface areas of the riders. BSA can be determined per rider based on height, weight and potentially age of the user. There are a number of formulas for calculating this value. In some embodiments, BSA is a value unique to each user and cannot be reused across the group. The BSA may have to be correlated to a cross sectional area in order to be applied to the generic drag equation.

Each of formation size 414, formation shape 416, participant velocities 418, wind 420, air density 422, and participant BSA 424 are used to compute a relative drag. In some embodiments, a relative drag can be expressed as:

Relative drag=(1/2)*(Density of fluid)*(velocitŷ2)*(Drag Coefficient)*(Cross Sectional Area) (Eq. 1)

Density of fluid depends on the fluid for example in water or air. In the case of air the density of dry air is computed based on based on pressure and temperature. In some embodiments, the density of humid air is calculated. Essentially both are derived from the ideal gas law. Temperature, humidity and pressure could be sampled through local sensors on the device or could be harvested based on a user's location.

Velocity refers to a speed of user relative to the fluid. This quantity would incorporate wind speed and GPS-derived speed.

Drag Coefficient refers to a quantity that can be derived by surveying datasets and using controlled wind speed and formation experiments with a diverse population of riders.

Cross Sectional Area (CSA) is directly proportional relationship to the body surface area where CSA=k*BSA where k would vary based on posture of the user in the fluid. For example, a user's posture can be detected via a phone or a watch. Then a CSA of the user can be calculated based on the detected posture.

Relative drag 404 can be used to determine a load adjustment factor 426. Relative drag 404 can translate into a percentage increase of a computed energy expenditure via the load adjustment factor 426. This adjustment factor 426 can be applied per epoch so it can be updated accordingly for speed, heading and formation changes.

The load adjustment factor 426 can be used to determine an energy expenditure 406, and in particular an adjusted energy expenditure 428 based on the relative drag 404. In general, the higher the relative drag 404, the higher the adjusted energy expenditure and the lower the relative drag 404, the lower the energy expenditure. For example, two cyclists, with otherwise similar biometrics and conditioning, traveling at the same velocity in a group can have different energy expenditures associated with their exercise session of the relative drag is different for each cyclist. The cyclist at the front of the group may experience positive drag and has to work harder and exert more energy at a given velocity, while a cyclist at the back of the group may experience negative drag (e.g., drafting or benefitting from the work of a cyclist in front) and has to work relatively less and exert less energy at a given velocity.

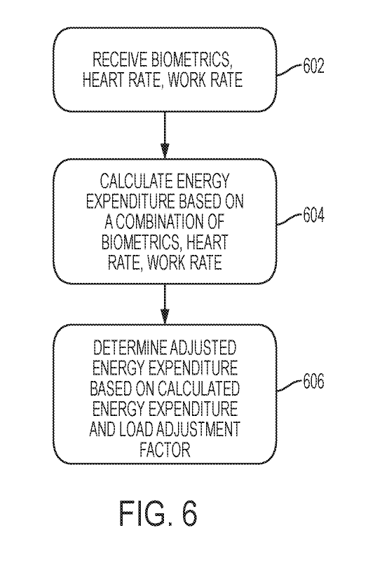

FIG. 6 is a flowchart showing a process for determining energy expenditure, according to some embodiments of the present disclosure.

Referring to step 602, a combination of biometrics, heart rate, and work rate are received from a combination of user inputs and sensors on the fitness tracking device 100 and the companion device 300.

Referring to step 604, energy expenditure is calculated based on a combination of biometrics, heart rate, and work rate. For example, various techniques exist for determining calorie estimation using measured heart rate or work rate. One technique is to use measured heart rate, maximum heart rate, and resting heart rate to determine a fraction of heart rate reserve (FHR) (referred to herein also as heart rate). Energy expenditure (EE), which is associated with calorie estimation, can be determined based on a calorimetry model using a calorimetry model with a parameterized function of FHR:

EE=VO₂max·f(FHR) (Eq. 2)

where VO2max refers to a maximal oxygen uptake.

Energy expenditure can also be determined as a function of work rate (WR):

EE=(A+B*load*WR)/(efficiency) (Eq. 3)

where load refers to a resistance associated with exercise, and efficiency refers to a ratio of work output to work input during exercise. The values “A” and “B” can be fixed or otherwise determined. Another expression of energy expenditure is metabolic rate. Metabolic rate may be expressed in Metabolic Equivalents of Task, or METs. METs indicates how many calories a “typical” individual burns per unit of body mass per unit of time. Calculating energy expenditure and METs is further described in U.S. Provisional Application No. 62/311,479, filed Apr. 7, 2016, and U.S. application Ser. No. 15/061,653, filed on Mar. 23, 2016, the contents of which are incorporated herein in their entireties.

Referring to step 606, an adjusted energy expenditure is determined based on the calculated energy expenditure and load adjustment factor. As described above, the load adjustment factor depends upon a relative drag associated with a user. Assuming a load adjustment factor, LA, the adjusted energy expenditure is: LA*EE.

Systems and methods are disclosed herein of determining a calorie expenditure value during group cycling. In some embodiments, the systems and methods include determining, by a processor of a fitness tracking device, a start of a cycling session associated with a user; measuring, by at least one sensor of the fitness tracking device, a location of the user, a Bluetooth® proximity value associated with a distance between the user and at least one other user, a wind speed and direction, air density, and a body surface area associated with the user and the least one other user; determining, by the processor of the fitness tracking device, relative position information associated with the user based on the measured location of the user and the Bluetooth® proximity value associated with a distance between the user and at least one other user, the relative position information including data associated with formation size, formation shape and velocities associated with the user and the at least one other user; determining, by the processor of the fitness tracking device, an amount of drag based on the relative position information, the wind speed and direction, the air density, and the body surface area associated with the user and the least one other user; calculating, by the processor of the fitness tracking device, a load adjustment factor based on the relative drag; and calculating, by the processor of the fitness tracking device, an adjusted energy expenditure based on the load adjustment factor.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of nonvolatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. A method for improving the accuracy of a wearable device while calculating an individual energy expenditure for a user participating in a group cycling session, the method comprising:
measuring, by a heart rate sensor of the wearable device, a heart rate of the user, wherein the heart rate sensor comprises a photoplethysmogram (PPG) sensor and the PPG sensor is configured to be worn adjacent to the user's skin;
calculating, by a processor circuit of the wearable device, an energy expenditure of the user based on at least the measured heart rate;
determining, by the processor circuit, a group formation size and a group formation shape based on a wireless-based proximity;
receiving, from an external source, a wind speed and direction;
determining, by a GPS module of the wearable device, the user's velocity;
determining, by the processor circuit, an air density based on an ambient temperature and an atmospheric pressure;
determining, by the processor circuit, a relative drag associated with the user based on the group formation size, the group formation shape, the user's velocity, the user's body surface area, the wind speed and direction, and the air density;
calculating, by the processor circuit, a load adjustment factor based on the determined relative drag;
determining, by the processor circuit, an updated energy expenditure based on the calculated energy expenditure and the load adjustment factor; and
outputting, by the processor circuit, the updated energy expenditure.

2. The method of claim 1, further comprising:
detecting, by a motion sensing module of the wearable device, the user's posture;
determining, by the processor circuit, a cross sectional area of the user based on the user's body surface area and the detected posture.

3. The method of claim 1, further comprising:
determining, by the processor circuit, a number and a relative position of nearby devices;
determining, by the processor circuit, the group formation size based on the number of nearby devices; and
determining, by the processor circuit, the group formation shape based on the relative position of nearby devices.

4. The method of claim 3, wherein determining a number of nearby devices comprises determining a number of devices that have successfully connected to the wearable device wirelessly.

5. The method of claim 3, wherein determining a number of nearby devices comprises determining a number of devices within a pre-defined distance from the wearable device.

6. The method of claim 3, wherein determining a relative position of nearby devices comprises performing time of flight calculations, measuring wireless signal strength of nearby devices, or using multiple directional antennas.

7. The method of claim 3, where determining a group formation shape comprises:
deriving, by the processor circuit, a map of devices from the relative position of nearby devices;
performing, by the processor circuit, a matching between the derived map of devices and one or more known group formation shapes; and
determining, by the processor circuit, a group formation shape based on a result of the matching.

8. The method of claim 7, wherein the known group formation shapes comprises a straight line, a cluster, echelon, single paceline, double paceline, circular paceline, or V formation.

9. A system for improving the accuracy of a wearable device while calculating an individual energy expenditure for a user participating in a group cycling session, the system comprising:
a heart rate sensor configured to measure a hear rate of the user, wherein the heart rate sensor comprises a photoplethysmogram (PPG) sensor and the PPG sensor is configured to be worn adjacent to the user's skin;
a GPS module configured to measure the user's location and velocity;
a wireless module configured to measure a wireless-based proximity of nearby devices; and
a processor circuit coupled to the heart rate sensor, the GPS module, and the wireless module and configured to execute instructions causing the processor circuit to:
calculate an energy expenditure based on at least the measured heart rate;
determine a group formation size and a group formation shape based on the measured wireless-based proximity;
determine an air density based on an ambient temperature and an air pressure;
receive a wind speed and direction from an external source;
determine a relative drag associated with the user based on the group formation size, the group formation shape, the user's velocity, the user's body surface area, the wind speed and direction, and the air density;
calculate a load adjustment factor based on the determined relative drag;
determine an updated energy expenditure based on the calculated energy expenditure and the load adjustment factor; and
output the updated energy expenditure.

10. The system of claim 9, wherein the instructions further cause the processor circuit to determine a cross sectional area of the user based on the user's body surface area and a posture of the user detected by a motion sensing module.

11. The system of claim 9, wherein the instructions further cause the processor circuit to:
determine a number and a relative position of nearby devices;
determine the group formation size based on the number of nearby devices; and
determine the group formation shape based on the relative position of nearby devices.

12. The system of claim 11, wherein the instructions further cause the processor circuit to determine a number of devices that have successfully connected to the wearable device wirelessly.

13. The system of claim 11, wherein the instructions further cause the processor circuit to determine a number of devices within a pre-defined distance from the wearable device.

14. The system of claim 11, wherein the instructions further cause the processor circuit to:
derive a map of devices from the relative position of nearby devices;
perform a matching between the derived map of devices and one or more known group formation shapes; and
determine a group formation shape based on a result of the matching.

15. The system of claim 14, wherein the known group formation shapes comprises a straight line, a cluster, echelon, single paceline, double paceline, circular paceline, or V formation.

16. A mobile device comprising the system of claim 9.
